(19) United States
(12) Patent Application Publication
(10) Pub. No.: US 2017/0188809 A1
(43) Pub. Date: Jul. 6, 2017

(54) SYSTEM AND METHOD FOR CENTRAL VISION ASSESSMENT AND TRACKING

(71) Applicant: Ram Peddada, Dothan, AL (US)

(72) Inventor: Ram Peddada, Dothan, AL (US)

(21) Appl. No.: 15/234,692

(22) Filed: Aug. 11, 2016

Related U.S. Application Data
(63) Continuation-in-part of application No. 14/986,681, filed on Jan. 2, 2016.

(51) Int. Cl. A61B 3/024; A61B 3/00

(52) U.S. Cl. CPC A61B 3/024; A61B 3/0025; A61B 3/0041; A61B 3/0033

(57) ABSTRACT

A system and method for assessing vision and tracking changes in vision is provided. A shape is presented on a display to a user who experiences partial or complete vision loss in the central or paracentral region of the visual field. The user manipulates the dimensions of the shape to correspond to a region of vision loss, and a metric is calculated to indicate the spatial extent of the region of vision loss. Metrics from previous assessments can be compared to track progression of visual impairment.

### CROSS REFERENCES

This application is a continuation-in-part of prior U.S. application Ser. No. 14/986,681, filed on Jan. 2, 2016, currently pending, which application is incorporated herein by reference.

### FIELD OF THE INVENTION

The present invention relates generally to systems and methods for assessing vision.

### BACKGROUND

Central or paracentral vision loss is a common visual condition that may be caused by a variety of retinal diseases such as age-related macular degeneration. A patient will typically experience some degree of visual impairment in or near the center of the visual field. The region of visual defect may be perceived by the patient as a distorted or blurry region that may range in appearance from grayish in color to completely dark. As the condition worsens, the region of visual defect may increase in size and/or become darker in color. If not treated properly, permanent vision loss may occur.

Assessing a patient's central or paracentral vision loss is typically done through basic screening charts such as an Amsler grid or by more sophisticated tests administered by trained optometrists or ophthalmologists. Sophisticated tests typically require costly equipment that must be operated by a trained professional. Additionally, many commonly used screening methods, such as an Amsler grid, provide a subjective, qualitative assessment of the patient's vision, which may not provide an accurate assessment of the patient's current state of visual impairment. In fact, according to one study, nearly 87% of scotomas (dark spots in central vision) were not detected by an Amsler grid test.

Once the patient has been diagnosed and the severity of vision impairment assessed, a variety of treatments may be administered to treat central or paracentral vision loss. For instance, injecting medication into the eye is generally considered the standard of care for treating central vision loss due to age-related macular degeneration. Injections are typically administered once per month and may cause the region of visual defect to decrease in size and/or degree of darkness. However, subjective vision assessments during the treatment period may not be effective in assessing and tracking the patient's progress. For instance, some proposed vision test methods may present a series of randomly shaped and randomly sized targets displayed in random locations in the visual field. The goal of such tests is to evaluate vision loss by determining which of the shapes the patient can or cannot see based on the size, shape, and location of each of the shapes. This type of test may be inefficient and inaccurate due to the randomness of the presentation and also may not provide a useful quantitative assessment of how the patient's vision may have changed since the previous assessment. Thus, this type of testing makes it difficult to accurately and objectively track a patient's subjective perception of progression when the region of visual defect changes in shape, size, and/or degree of darkness over a period of time. The tracking of subjective perception of progression is an important component in the treatment of many of the retinal diseases since this is what will alert a patient to schedule an appointment with a physician for any additional treatment.

Accordingly, a need exists in the art for a system and method for quantitatively assessing vision loss and for tracking changes in vision loss over a period of time. Furthermore, a need exists in the art for a system and method that allows patients to quantitatively and independently monitor their own vision functioning to provide better assessment of patient progress during time periods between treatments for visual impairment conditions.

### SUMMARY

In one aspect, the present disclosure relates to a system and a method for assessing central and paracentral vision defects and tracking changes in central and paracentral vision. In a preferred embodiment of the method of the present disclosure, a shape is presented to a user on a display screen, which is operably connected to a computer. The computer is configured to allow the user to manipulate the dimensions of the shape via user input. The user then manipulates the dimensions of the shape such that the shape corresponds to the spatial extent of a region of vision loss. Based on a measurement of the size of the shape, the computer is configured to calculate a quantitative spatial metric indicating the spatial extent of the region of vision loss.

For instance, in one embodiment, the shape is a circle that is relatively small such that the circle is at least partially obscured as perceived by the user due to the region of visual defect. The user then looks towards a centrally located target point and slowly increases the size of the circle until the circle circumscribes the region of visual defect. At this point, the increasing of the size of the circle is stopped. The computer is configured to take a measurement of the size of the circle and calculate a quantitative spatial metric indicating the spatial extent of the region of vision loss based on the measurement. The quantitative metric is stored in a database. The method may be repeated at spaced time intervals to assess changes over a period of time and to create a quantitative record of changes in the spatial extent of vision loss.

In a preferred embodiment, the shape presented to the user has a color, which is preferably gray. The computer is configured to allow the user to vary the value of the color via user input. The user then varies the value of the color to make the color darker or lighter such that the color of the shape corresponds to the color of the region of vision loss as perceived by the user. Thus, the color of the shape is varied until the shade of gray of the shape approximates the shade of gray perceived by the user in the region of the visual defect. The final color selected by the user has a color value which is used to calculate a quantitative color metric that indicates the degree of visual impairment in the region of vision loss. The quantitative color metric is stored in a database, and the assessment may be repeated over a period of time to create a quantitative record of changes to the user's vision over the duration of the time period. Generally, a darker color indicates a greater degree of visual impairment than a lighter color. Thus, if the shade of gray selected by the user in subsequent tests becomes lighter in color, this result would generally indicate an improvement in vision even if the spatial extent of the vision loss stays approximately the same in subsequent tests.

The vision assessments methods described herein may be performed by a trained professional at a healthcare facility or alternatively by a patient at home having access to a computer system configured for implementing the methods described herein. The method of vision assessment is particularly beneficial to patients who may want to proactively monitor their vision between visits to an optometrist or ophthalmologist. For instance, patients diagnosed with central vision loss often receive treatment of monthly injections of medication into the eye. Between monthly visits, it may be advantageous for the patient to assess his or her vision more frequently, such as daily or weekly, to track improvement of the visual impairment or to determine if the impairment is worsening. In some instances, a patient performing periodic self-assessments may see the extent of the visual defect remain at approximately the same size but may see the color of the region of defect becoming lighter, which generally indicates that the treatment is working. In other instances, the patient may see improvement and then worsening before the next treatment, which likely indicates that the treatments should be administered more frequently. In cases of significant worsening of the impairment, periodic self-assessment will prompt the patient to alert a retina specialist. The quantitative nature of the assessment methods described herein provide a simple and consistent result for self-assessment such that the progression of the impairment can be accurately tracked by recording a quantitative metric that objectively assesses a patient's subjective perception of a region of visual defect.

Another aspect of the disclosure includes a system configured for assessing central or paracentral vision loss. In one embodiment, the system comprises a display screen and a computer operably connected to the display screen. The computer is configured to present a shape to a user on the display screen and to allow the user to manipulate the dimensions of the shape via user input such that the shape corresponds to the spatial extent of a region of vision loss of the user. The computer is further configured to calculate a quantitative spatial metric indicating the spatial extent of the region of vision loss based on a measurement of the size of the shape.

The foregoing summary has outlined some features of the systems and methods of the present disclosure so that those skilled in the pertinent art may better understand the detailed description that follows. Additional features that form the subject of the claims will be described hereinafter. Those skilled in the pertinent art should appreciate that they can readily utilize these features for designing or modifying other structures for carrying out the same purposes of the systems and methods disclosed herein. Those skilled in the pertinent art should also realize that such equivalent designs or modifications do not depart from the scope of the systems and methods of the present disclosure.

### DESCRIPTION OF THE DRAWINGS

These and other features, aspects, and advantages will become better understood with regard to the following description, appended claims, and accompanying drawings where:

FIG. 1 is a perspective view of an interactive vision assessment system and method being utilized by a use in accordance with the present disclosure.

FIG. 2A illustrates an image of a reference circle for vision assessment in accordance with the present disclosure.

FIG. 2B illustrates an image of a reference circle for vision assessment in accordance with the present disclosure.

FIG. 2C illustrates an image of a reference circle for vision assessment in accordance with the present disclosure.

FIG. 2D illustrates an image of reference circles for vision assessment in accordance with the present disclosure, including a region of visual defect as perceived by a patient superimposed onto the image.

FIG. 3A illustrates an image of a reference ellipse for vision assessment in accordance with the present disclosure.

FIG. 3B illustrates an image of a reference ellipse for vision assessment in accordance with the present disclosure.

FIG. 3C illustrates an image of a reference ellipse for vision assessment in accordance with the present disclosure.

FIG. 3D illustrates an image of a reference ellipse for vision assessment in accordance with the present disclosure.

FIG. 3E illustrates an image of reference ellipses for vision assessment in accordance with the present disclosure, including a region of visual defect as perceived by a patient superimposed onto the image.

FIG. 4A illustrates an irregularly shaped reference image for vision assessment in accordance with the present disclosure.

FIG. 4B illustrates an irregularly shaped reference image for vision assessment in accordance with the present disclosure.

FIG. 5 illustrates an irregularly shaped reference image for vision assessment in accordance with the present disclosure.

FIG. 6 illustrates a flow chart showing method steps for vision assessment in accordance with the present disclosure.

### DETAILED DESCRIPTION

In the Summary above and in this Detailed Description, and the claims below, and in the accompanying drawings, reference is made to particular features, including method steps, of the invention. It is to be understood that the disclosure of the invention in this specification includes all possible combinations of such particular features. For example, where a particular feature is disclosed in the context of a particular aspect or embodiment of the invention, or a particular claim, that feature can also be used, to the extent possible, in combination with or in the context of other particular aspects of the embodiments of the invention, and in the invention generally.

The term “comprises” and grammatical equivalents thereof are used herein to mean that other components, steps, etc. are optionally present. For example, an article “comprising” components A, B, and C can contain only components A, B, and C, or can contain not only components A, B, and C, but also one or more other components.

Where reference is made herein to a method comprising two or more defined steps, the defined steps can be carried out in any order or simultaneously (except where the context excludes that possibility), and the method can include one or more other steps which are carried out before any of the defined steps, between two of the defined steps, or after all the defined steps (except where the context excludes that possibility).

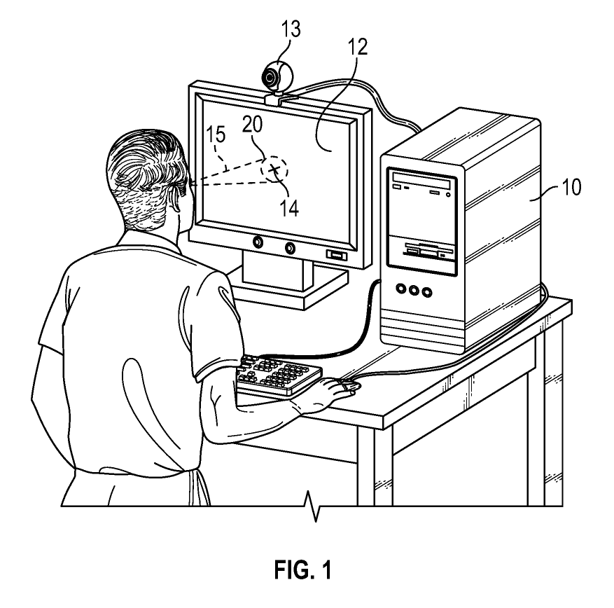

In one aspect, a method for assessing central and paracentral vision defects and tracking changes in central and paracentral vision is provided. FIG. 1 shows a vision testing system that may be used in accordance with the disclosed method. A user experiencing central or paracentral vision loss looks toward a target point 14 on a display screen 12. The target point 14 is preferably located near the center of the display 12. A shape, such as a circle 20, is presented to the user on the display 12. The user manipulates the dimensions of the shape so that the shape corresponds to the spatial extent of a region of vision loss that is perceived by the user. Based on a measurement of the size of the adjusted shape, a quantitative spatial metric is calculated. The metric indicates the spatial extent of the region of vision loss. The method may be performed by a user at home as a self-assessment method. By providing the user with a quantitative measure of the spatial extent of vision loss, the user can track the progression of his or her vision over a period of time.

As used herein, a region of vision loss or visual defect refers to a region in the visual filed in which a patient experiences some degree of visual impairment up to total loss of vision within the affected region. The visual impairment may include distorted or blurry vision and may range in appearance from grayish in color to completely dark. In addition, as used herein, element number 20 refers generally to any circle presented to the user for vision assessment, and element numbers 20a, 20b, 20c, 20d, and 20e refer to specific examples of displayed circles shown in various illustrative embodiments.

FIG. 1 depicts an illustrative example of a computer system that may be used to perform the methods disclosed herein. The system is configured to allow the user to manipulate the dimensions of a shape while viewing the shape in order to perform a quantitative assessment of the user's vision based on the manipulated shape. Those skilled in the art should appreciate that any type of suitable computer system may be utilized to implement the methods of the present disclosure. For instance, computing systems that may be utilized may include, but are not limited to, desktop computers, laptop computers, game consoles, or handheld computing devices such as a smartphone, tablet computer, personal digital assistant, or other types of handheld computing devices, or any computing device specifically designed to perform the present methods. The display screen 12 may include any suitable screen such as a desktop display, laptop display, television screen, smartphone or tablet screen, or any other screen suitable for performing a vision test, such as a projector screen. In a preferred embodiment, a relatively large screen is utilized to provide greater area for the user to manipulate the dimensions of the shape presented to the user. In one embodiment, the display 12 may comprise a touchscreen for controlling the functioning of the assessment methods.

As shown in FIG. 1, the vision testing system comprises a display screen 12 operably connected to a computer 10 such that the display screen 12 visually displays information resulting from performing steps of the disclosed methods. The computer 10 is configured to present the shape to the user on the display screen 12 and to allow the user to manipulate the dimensions of the shape via user input. The user manipulates the dimensions of the shape such that the manipulated shape corresponds to the spatial extent of the region of vision loss perceived by the user. The computer 10 changes the dimensions of the presented shape according to the user input, which is preferably received via a graphical user interface. The computer 10 is further configured to calculate the quantitative spatial metric for quantitatively assessing the vision loss of the user. In a preferred embodiment, the system further comprises a database for storing assessment metrics and other associated data. Thus, the user may perform a set of vision assessments at spaced time intervals to create a quantitative record of changes to the user's vision over a period of time. The quantitative record may include metrics related to the spatial extent of the region of vision loss, the degree of visual impairment within the region, or both.

When using the system for vision assessment, it may be beneficial for the user to maintain an appropriate viewing distance 15 from the display screen 12 in order to maintain consistent results in the assessment. In one embodiment, a camera 13 may assist the user in ensuring that the viewing distance 15 is appropriate. The user should establish an appropriate viewing distance 15 the first time that the user uses the system. At an appropriate viewing distance 15, the user should be able to clearly see the shape being presented to the user on the display screen 12. The system may then be calibrated to set the distance 15. For instance, the camera 13 may be configured to show an image of the user's face on the display 12 with markings on the screen 12 to indicate an outline of the user's face, horizontal lines indicating upper and lower boundaries of the user's face, vertical lines indicating left and right side boundaries of the user's face, any combination of such markings, or any other markings suitable to indicate the positioning of the user's face on the display screen 12 relative to the camera 13. The system may then be calibrated to show these markings each time the system is used so that the user can position his or her face at the appropriate distance based on the location of the markings superimposed onto a live image of the user's face on the display screen 12. In this manner, a consistent distance 15 can be maintained each time the assessment methods are carried out to achieve consistent results.

In another embodiment, a chin rest may be employed to maintain an appropriate viewing distance 15. Once an appropriate distance has been established, the chin rest can be fixed in the appropriate location and used for subsequent assessments. In addition, a chin rest may help the user keep his or her vision steady during the vision assessment. Those skilled in the art should appreciate that any suitable method for maintaining an appropriate viewing distance may be utilized and fall within the scope of the present disclosure.

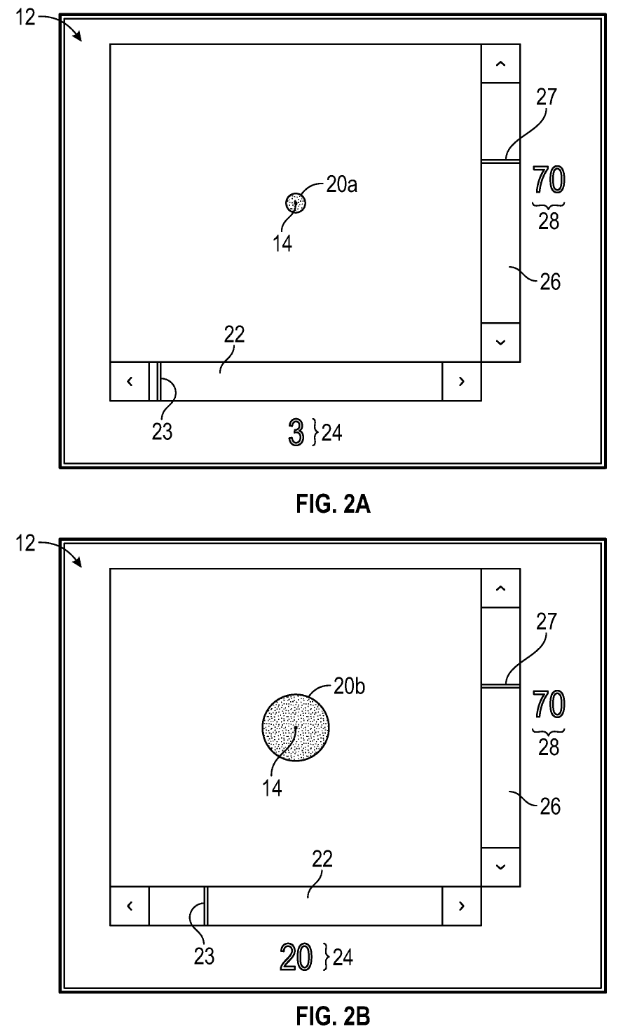

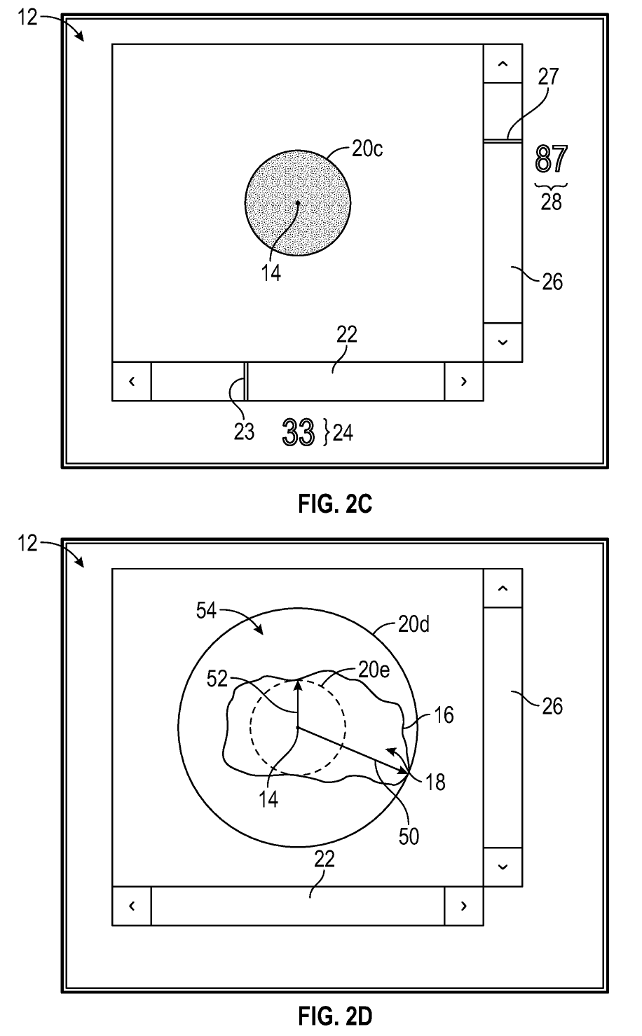

FIGS. 2A-2D show a preferred embodiment of a display screen 12 that may be used in accordance with the methods of the present disclosure. In this embodiment, the shape presented to the user on the display 12 is a circle 20. A target point 14 is located at the center of the circle 20. In this embodiment, the target point 14 is represented by a dot but may alternatively be represented by a small “x” shape or a similar type visual target. The computer 10 is configured to allow the user to manipulate the dimensions of the circle 20 as it appears on the display screen 12. In this embodiment, the dimensions of the circle 20 are manipulated by increasing or decreasing the size of the circle 20 according to user input.

A number of methods for manipulating the dimensions of the circle 20 may be utilized to practice the methods disclosed herein. In a preferred embodiment, user inputs are received via a graphical user interface. As shown in FIGS. 2A-2D, the interface may comprise an adjustment bar 22 with a slider 23 configured for varying the size of the circle 20. The adjustment bar 22 is presented to the user on the display screen 12. Utilizing a computer mouse, the user can click and drag the slider 23 along the adjustment bar 22 to vary the size of the circle 20. Alternatively, the adjustment bar may have an arrow at each end that can be clicked with the mouse to adjust the size of the circle 20. In this embodiment, dragging the slider 23 to the right or clicking the arrow pointing right increases the size of the circle 20, and dragging the slider 23 to the left or clicking the arrow pointing left decreases the size of the circle 20. For example, FIGS. 2A-2C show circles 20a, 20b, 20c of increasing size. As shown in FIG. 2A, the circle 20a is relatively small and the slider 23 is relatively far to the left of the adjustment bar 22. In FIG. 2B, the slider 23 has been moved farther to the right along the adjustment bar 22, and the circle 20b is larger. In FIG. 2C, the size of the circle 20c has been increased further by moving the slider 23 farther to the right on the adjustment bar 22. In this embodiment, the size adjustment bar 22 is positioned horizontally along the bottom of the display 12 but may be positioned in any suitable location on the display 12.

Of course, other types of user inputs for varying the size of the circle 20 may be utilized. For example, a touchscreen may be utilized that allows the user to expand or contract the size of the circle 20 by touching the screen with two fingers and moving the fingers farther apart or closer together. In other embodiments, the size of the circle 20 may be varied by using keyboard arrows, a touchpad, a track ball, a joystick, or by turning a knob. In another embodiment, the system may comprise a microphone configured to accept voice commands, and the user may use voice commands to vary the size of the circle. In yet another embodiment, the vision assessment may be administered by another person, such as an optometrist or ophthalmologist or any individual assisting the user, and the size of the circle may be varied by the user by instructing the other person to change the size of the circle by any suitable method, such as the previously described methods.

FIG. 2D illustrates an example of a display screen 12 that a user may see during vision assessment. FIG. 2D shows a region 18 of visual defect superimposed on the image of the screen 12 as perceived by the user. The region 18 of visual defect in this case, as is typical of such visual defects, has an irregular shape located in the central or paracentral regions of the visual field. As shown in FIG. 2D, the region 18 of vision loss is represented by an irregularly shaped outline 16 having an internal area within the boundaries of the outline 16. To the user, this region 18 may have the appearance of a blurry, grayish area or may appear completely dark. In addition, the region 18 of vision loss may change in size and/or color over a period of time, depending on whether the visual defect is improving or worsening. The edges of the region 18 as perceived by the user may appear blurred and may not be well defined, but for ease of illustration the region is represented in FIG. 2D by a well-defined outline 16. Also for ease of illustration, the interior portion of shapes, including the region of visual defect, is shown in FIG. 2D in white.

To practice the vision assessment method of the embodiment shown in FIG. 2D, the user would first look toward the target point 14 at the center of the screen 12. Because the target point 14 may be obscured by the region 18 of vision loss, the user may temporarily adjust his sight lines to determine the location of the target point 14 and then center his focus around this point. Next, the user varies the size of the circle 20 such that the circle corresponds to the spatial extent of the region of vision loss perceived by the user. In one embodiment shown in FIG. 2D, which utilizes a circle 20 as the shape presented to the user, the circle 20d corresponds to the spatial extent of the region 18 of vision loss when the circle 20d generally circumscribes the region 18 of vision loss. In an alternative embodiment, also shown in FIG. 2D, the circle 20e corresponds to the spatial extent of the region 18 of vision loss when the circle 20e is generally circumscribed by the region 18 of vision loss. In this case, the embodiment of the method utilizing circle 20d is preferred because circle 20d corresponds to the fullest extent of the region 18 of vision loss due to circle 20d being approximately tangential to the farthest point of the region 18 of vision loss from the centrally located target point 14.

Because the boundary 16 of the region 18 of vision loss may appear blurred to the user, the final size of the circle 20d selected by the user should be as small as possible with the user being able to see the entire outline of the circle 20d such that the circle 20d generally circumscribes the region 18 of vision loss. To make it easier for the user to see the outline of the circle, the outline of the circle may use a line having a different color or different line weight than the line shown in the figures, or may use any other suitable line quality to help the user see the circle. In the embodiment utilizing circle 20e, the final size of the circle 20e selected by the user should be as small as possible with the user still being able to see some portion of the outline of the circle 20e such that the circle 20e is generally circumscribed by the region 18 of vision loss. In one embodiment, the assessment may begin with a relatively small circle 20 such as circle 20a shown in FIG. 2A, and the user can increase the size of the circle 20 to reach the final size. Alternatively, the assessment may begin with a relatively large circle 20, and the user can decrease the size of the circle 20 to reach the final size.

Once the final circle 20 of appropriate size has been selected by the user, a quantitative spatial metric 24 is calculated based on a measurement of the size of the circle 20. The spatial metric 24 indicates the spatial extent of the region 18 of vision loss by providing a quantitative measurement corresponding to the region 18 of vision loss. A number of different measurements of the size of the shape may be utilized to calculate the quantitative spatial metric 24. In the embodiment utilizing a circle 20 as the shape presented to the user, the measurement utilized is preferably a measure of the radius 50 of a circle 20d circumscribing the region 18 of vision loss, which indicates the farthest point of the region 18 of vision loss from the target point 14. Other measurements which may be utilized include the diameter, the circumference, or the area of the circle. In the embodiment utilizing a circle 20e circumscribed by the region 18 of vision loss, radius 52 is preferably utilized as the measurement.

The system is configured to calculate the quantitative spatial metric 24 based on the final size of the circle 20 selected by the user. The system is preferably further configured to display the quantitative spatial metric 24 to the user on the display 12, as shown in FIGS. 2A-2C. It should be noted that the numeric values shown, “3”, “20”, and “33”, are for illustrative purposes only to show relative differences in the size of the circles 20a, 20b, and 20c, respectively. These circles may not be drawn to scale, and the numeric values shown are relative values that do not represent a particular unit of measurement, though any suitable unit of measurement may be utilized.

In other embodiments, shapes other than circles may be utilized, such as polygonal shapes having sides of equal length. In such embodiments, the size of the shape is varied in such a manner that the length of each side changes but remains equal to the length of the other sides.

In a preferred embodiment, the circle 20 or other shape presented to the user has a color, and the system is further configured to allow the user to vary the value of the color of the circle 20 via user input. In a preferred embodiment, the color of the shape is gray, and the color value of the circle 20 can be varied such that the color ranges between white and black. In alternative embodiments, other colors or hues may be utilized, and the value of those colors or hues may be varied. A number of user input methods for varying the color value of the circle 20 may be utilized to practice the methods disclosed herein. In a preferred embodiment, an interface comprising an adjustment bar 26 with a slider 27 is configured for varying the color value of the circle 20. The adjustment bar 26 is presented to the user on the display 12. Utilizing a computer mouse, the user can click and drag the slider 27 along the adjustment bar 26 to vary the color of the circle 20. Alternatively, the adjustment bar may have an arrow at each end that can be clicked with the mouse to adjust the color of the circle 20. In the embodiment shown in FIGS. 2A-2C, the color adjustment bar 26 is positioned vertically on the right side of the display 12 but may be positioned in any suitable location on the display 12. In this embodiment, dragging the slider 27 upward or clicking the arrow pointing upward makes the color of the circle 20 darker, and dragging the slider 27 downward or clicking the arrow pointing downward makes the color of the circle 20 lighter. For instance, circles 20a and 20b shown in FIGS. 2A and 2B, respectively, have the same color value, as indicated by a color metric 28, which is discussed in detail below. Circle 20c shown in FIG. 2C has a darker shade of gray, as indicated by the color metric 28 having a higher numeric value and the slider 27 being in a higher position than in FIGS. 2A and 2C.

Of course, other methods for varying the color value of the circle 20 may be utilized. For example, in other embodiments, the color value of the circle 20 may be varied by using a touchscreen, a touchpad, keyboard arrows, a track ball, a joystick, or by turning a knob. In another embodiment, the system may comprise a microphone configured to accept voice commands, and the user may use voice commands to vary the color value of the circle. In yet another embodiment, the vision assessment may be administered by another person, such as an optometrist or ophthalmologist or any individual assisting the user, and the color value of the circle may be varied by the user by instructing the other person to change the color value of the circle by any suitable method, such as the previously described methods. Alternately, a non-computer version may include a series of drawings on paper or similar material depicting different shapes with a gray interior having color values varying from white to black shades.

In accordance with a preferred embodiment of the disclosed method, the user varies the value of the color via user input to make the color darker or lighter such that the color of the circle 20 corresponds to the color of the region 18 of vision loss as perceived by the user. In a preferred embodiment in which the color is gray, the value of the color of the circle 20 is varied until the shade of gray inside the circle 20 approximates a shade of gray perceived by the user in the region 18 of the vision loss. For example, in the embodiment shown in FIG. 2D, circle 20d has an area 54, which partially lies outside the boundary 16 of the region 18 of vision loss. Thus, the user varies the value of the color of the circle 20d until the shade of gray within area 54 approximates the shade of gray within region 18, which is the region of vision loss as perceived by the user. In the embodiment utilizing a circle 20e circumscribed by the region 18 of vision loss, after completing the portion of the assessment for assessing the spatial extent of the vision loss, the user can increase the size of the circle to make at least a portion of the area of the circle fall outside the region 18 of vision loss so that the user can see an inside portion of the circle for varying the color value of the circle. The computer 10 is configured to vary the color value according to the user input.

The final color selected by the user has a color value which is used to calculate a quantitative color metric 28 that indicates the degree of visual impairment within the region 18 of vision loss. In a preferred embodiment, the color metric 28 has a value ranging from 0-100, with the value “0” corresponding to white and the value “100” corresponding to black. Those skilled in the art should appreciate that other measurements indicating a color value may be utilized without departing from the scope of the present disclosure.

The system is configured to calculate the quantitative color metric 28 based on the final color of the circle 20 selected by the user. The system is preferably further configured to display the quantitative color metric 28 to the user on the display 12, as shown in FIGS. 2A-2C. It should be noted that the numeric values shown, “70”, “70”, and “87”, are for illustrative purposes only to show relative differences in the color of the circles 20a, 20b, and 20c, respectively. These numeric values may not accurately represent the color of the circles as illustrated in the drawings.

The quantitative color metric 28 is stored in a database, and the assessment may be repeated over a period of time to create a quantitative record of changes to the user's vision over the duration of the time period. Generally, a darker color indicates a greater degree of visual impairment than a lighter color. Thus, if the shade of gray selected by the user in subsequent tests becomes lighter in color, this result would generally indicate an improvement in vision even if the spatial extent of the vision loss remains approximately the same in subsequent tests.

In a preferred embodiment of the method of vision assessment disclosed herein, the user first manipulates the dimensions of the shape to assess the spatial extent of the region of vision loss. Once this portion of the assessment is complete and a quantitative spatial metric has been calculated, the user proceeds to vary the color value of the shape to assess the degree of visual impairment experienced within the region of vision loss. However, the assessment of the spatial extent of vision loss and the assessment of the degree of visual impairment may be performed independently or in the reverse order.

The system is configured to store both the spatial metric and the color metric for each administered test in a database. In a preferred embodiment, both types of assessments are performed in each individual test, and the test may be repeated at spaced time intervals over a duration of time such that one or both metrics for each iteration is stored in the database to create a quantitative record of changes to the user's vision. Thus, each vision assessment preferably comprises two individual quantitative metrics, which are stored in the database. Other associated data may also be stored in the database. For instance, the system is preferably configured to store user identification information with the stored metrics and any other relevant user data. The system may also be configured to date stamp and time stamp the calculated metrics to indicate the date and time of the assessment. Geographic location data may also be attached to each quantitative metric and stored in the database.

Storing the quantitative metrics and associated data in the database allows the user to create a quantitative record of changes to the user's vision over a period of time in which the user's vision has been assessed at spaced intervals. Assessments may be performed by a professional eye care provider or by the user at home using a computer system configured for performing the assessment. Thus, a user can monitor his or her vision between visits to an eye care professional. The quantitative metrics produced by the assessment methods disclosed herein provide an objective assessment of the patient's subjective perception to provide a baseline for assessing any changes to the user's vision with respect to changes in the spatial extent of vision loss or changes in the degree of visual impairment in a quantitative manner.

Patients diagnosed with central vision loss often receive treatment of monthly injections of medication into the eye. Between monthly visits, the patient may self-assess his or her vision and quantitatively track the progression of the visual impairment to determine if the treatment is working. In some instances, a patient performing periodic self-assessments may see the extent of the visual defect remain at approximately the same size but may see the color of the region of defect becoming lighter, which generally indicates that the treatment is working. In other instances, the patient may see improvement and then worsening before the next treatment, which likely indicates that the treatments should be administered more frequently. In cases of significant worsening of the impairment, periodic self-assessment according to the methods described herein will prompt the patient to immediately alert a retina specialist. In addition, results of periodic assessments may be shared with the patient's eye care professional so that the professional can monitor progress without the need for appointments.

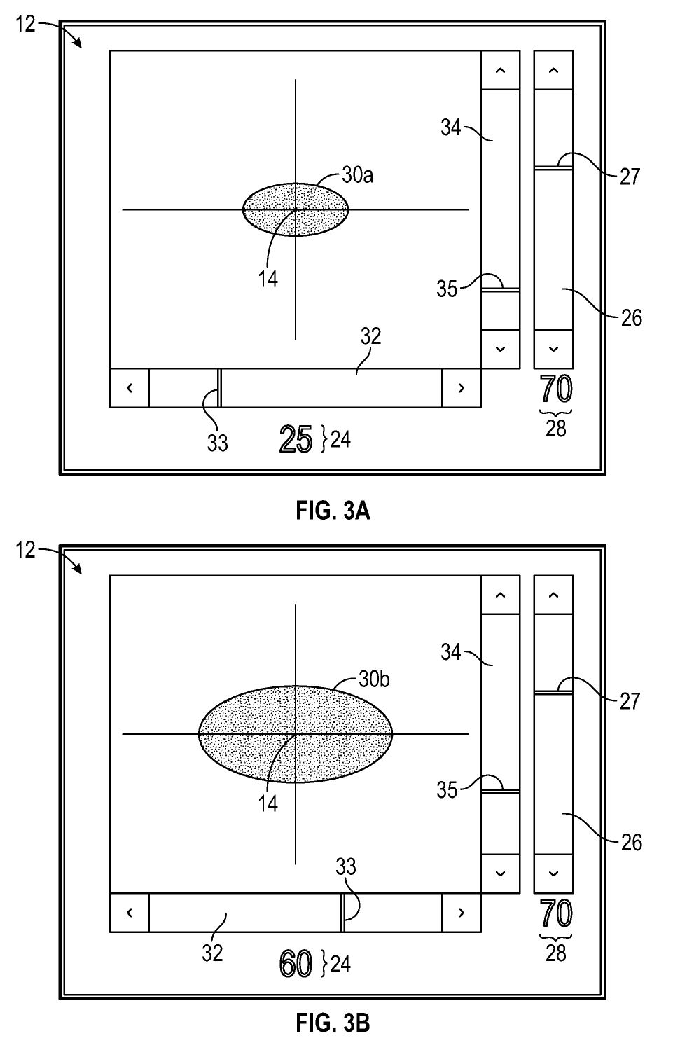

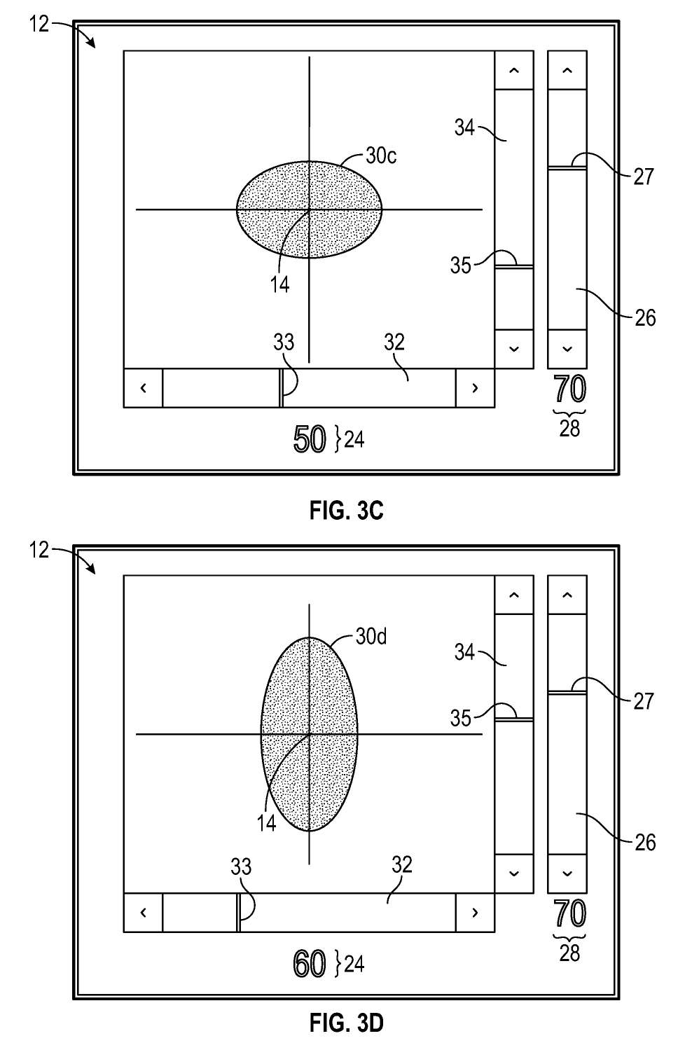

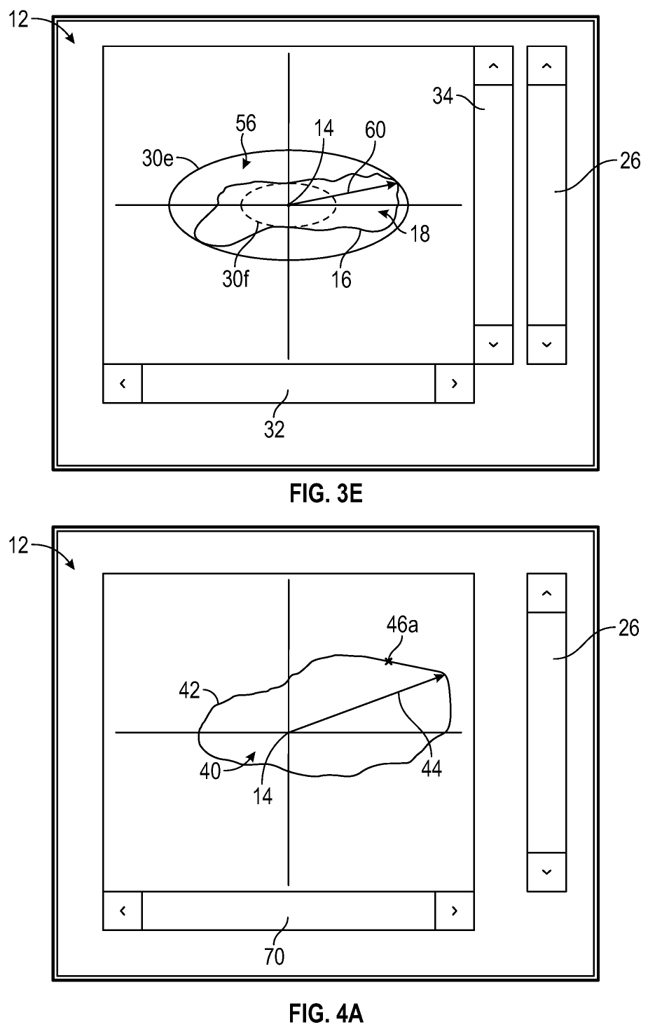

FIGS. 3A-3E show an embodiment in which the shape presented to the user on the display 12 is an ellipse 30. As used herein, element number 30 refers generally to any elliptical shape that is presented to the user for vision assessment and that has dimensions which can be varied on two axes. Element numbers 30a, 30b, 30c, 30d, 30e, and 30f refer to specific examples of displayed ellipses shown in various illustrative embodiments. In some cases, it may be beneficial to use an ellipse 30 rather than a circle 20 if the dimensions of the ellipse 30 can be manipulated in such a way that the ellipse 30 better conforms to the shape of the region 18 of visual defect. However, in some cases the simplicity of varying the size of a circle 20 may be preferred for patients performing self-assessments at home.

FIGS. 3A-3E show an embodiment of a display screen 12 that may be used in accordance with the methods of the present disclosure. In this embodiment, a generally horizontal x-axis and a generally vertical y-axis are shown. A target point 14 is located where the axes cross and an ellipse 30 is shown with the target point 14 generally at the center of the ellipse 30. The computer 10 is configured to allow the user to manipulate the dimensions of the ellipse 30 along the x-axis and the y-axis to form a new elliptical shape 30 having different dimensions. In this embodiment, the dimensions of the ellipse 30 are varied by increasing or decreasing the diameter of the ellipse 30 along the x-axis, the y-axis, or both axes, according to user input. In other embodiments, the dimensions of the shape may be varied along more than two axes. The computer 10 is configured for changing the dimensions of the ellipse 30 according to the user input.

In this embodiment, the dimensions of the ellipse 30 are preferably varied utilizing an interface comprising two adjustment bars 32, 34, as shown in FIGS. 3A-3E, though any suitable method of varying the dimensions may be utilized, including those previously discussed. An x-axis adjustment bar 32 with a slider 33 is presented to the user on the display 12. Utilizing a computer mouse, the user can click and drag the slider 33 along the adjustment bar 32 to vary the length of the diameter of the ellipse 30 along the x-axis. The y-axis adjustment bar 34 works in a similar manner. In this embodiment, the x-axis adjustment bar 32 is positioned horizontally along the bottom of the display 12 and the y-axis adjustment bar 34 is positioned vertically on the right side of the display 12, but these size adjustment bars may be positioned in any suitable location on the display 12. On the x-axis adjustment bar 32, dragging the slider 33 to the right increases the length of diameter along the x-axis, and dragging the slider 33 to the left decreases the length of the diameter. Similarly, on the y-axis adjustment bar 34, dragging the slider 35 upward increases the length of diameter along the y-axis, and dragging the slider 35 downward decreases the length of the diameter. Preferably, each size adjustment bar 32, 34 may have an arrow at each end that can be clicked with the mouse to adjust the length of the diameter of the ellipse 30 along each respective axis. For example, FIGS. 3A-3D show ellipses 30a, 30b, 30c, 30d of varying dimensions along the x and y-axes. In these examples, the ellipses 30 may be elongated along the x-axis or along the y-axis, as shown in FIG. 3D. In some instances, depending on the general shape of the region of visual defect of the user, the diameter along both axes may be approximately equal such that a circle is formed.

FIG. 3E illustrates an example of a display screen 12 that a user may see during vision assessment. FIG. 3E shows an irregularly shaped region 18 of vision loss superimposed on the image of the screen 12 as perceived by the user. The region 18 is represented by an outline 16 generally defining the boundaries of the region 18, which may have a blurry, grayish appearance or may appear completely dark.

To practice the vision assessment method of the embodiment shown in FIG. 3E, the user would first look toward the target point 14. Because the target point 14 may be obscured by the region 18 of vision loss, the user may temporarily adjust his sight lines to determine the location of the target point 14 and then center his focus around this point. Next, the user varies the dimensions of the ellipse 30 along each axis as necessary such that the ellipse corresponds to the spatial extent of the region of vision loss perceived by the user. In one embodiment shown in FIG. 3E, the ellipse 30e corresponds to the spatial extent of the region 18 of vision loss when the ellipse 30e generally circumscribes the region 18 of vision loss. To circumscribe the region of vision loss, the ellipse 30e is preferably sized such that it is approximately tangential to the region 18 of vision loss in at least two locations, as shown in FIG. 3E. The final dimensions of the ellipse 30e selected by the user should be as small as possible with the user being able to see the entire outline of the ellipse 30e such that the ellipse 30e generally circumscribes the region 18 of vision loss. In another embodiment shown in FIG. 3E, the ellipse 30f corresponds to the spatial extent of the region 18 of vision loss when the ellipse 30f is generally circumscribed by the region 18 of vision loss. In the embodiment utilizing ellipse 30f, the final size of the ellipse 30f selected by the user should be as small as possible with the user still being able to see some portion of the outline of the ellipse 30f such that the ellipse 30f is generally circumscribed by the region 18 of vision loss. Ellipse 30f is preferably sized such that it is approximately tangential to the region 18 of vision loss in at least two locations, and so at least two portions of the outline of the ellipse 30f should be visible to the user. In this case, the embodiment of the method utilizing ellipse 30e is preferred because ellipse 30e is approximately tangential to the region of vision loss in two locations, including the location of the farthest point of the region 18 of vision loss from the centrally located target point 14.

In this embodiment, the system is configured such that when the user varies the length of the diameter of the ellipse 30 along the x-axis or y-axis, the outline of the ellipse is automatically adjusted to maintain an elliptical shape during adjustments of the diameter along either axis.

Once the final ellipse 30 of appropriate dimensions has been selected by the user, a quantitative spatial metric 24 is calculated based on a measurement of the size of the ellipse 30. The spatial metric 24 indicates the spatial extent of the region 18 of vision loss by providing a quantitative measurement corresponding to the region 18 of vision loss. A number of different measurements of the size of the shape may be utilized to calculate the quantitative spatial metric 24. In the embodiment utilizing an ellipse 30 as the shape presented to the user, the measurement utilized is preferably a mean length of a plurality of radii of the ellipse 30 measured at angular increments such as one-degree increments. Other measurements which may be utilized include the longest radius or diameter of the ellipse, the circumference, or the area of the ellipse.

The system is configured to calculate the quantitative spatial metric 24 based on the final dimensions of the ellipse 30 selected by the user. The system is preferably further configured to display the quantitative spatial metric 24 to the user on the display 12, as shown in FIGS. 3A-3D. It should be noted that the numeric values shown, “25”, “60”, “50”, and “60”, are for illustrative purposes only to show relative differences in the dimensions of the ellipses 30a, 30b, 30c, and 30d, respectively. These ellipses may not be drawn to scale, and the numeric values shown are relative values that do not represent a particular unit of measurement, though any suitable unit of measurement may be utilized.

In a preferred embodiment, the ellipse 30 presented to the user has a color, as in previous embodiments, and the system is configured to allow the user to vary the value of the color of the ellipse 30 so that the color of the ellipse 30 corresponds to the color of the region of vision loss as perceived by the user. For example, the color value of area 56 within eclipse 30e shown in FIG. 3E can be varied such that the color approximates the color perceived by the user within the region 18 of vision loss. The color of the ellipse 30 is preferably varied using a color adjustment bar 26 as shown in FIGS. 3A-3E. A quantitative color metric 28 is calculated and displayed to the user.

In other embodiments, a shape other than an ellipse may be utilized to vary the dimensions of the shape along two axes. For instance, a rectangle could be utilized wherein the dimensions of the rectangle could be independently varied along an x-axis and a y-axis.

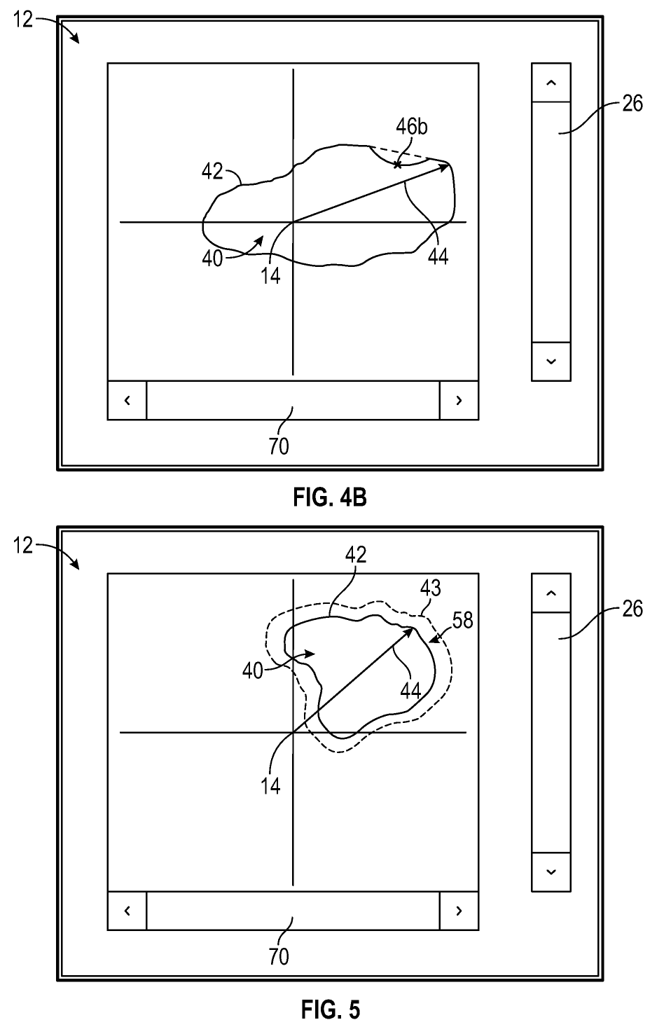

In another embodiment, the system is configured to allow the user to manipulate the dimensions of an originally presented shape, which may be a circle 20, an ellipse 30, or another suitable shape, by manipulating the outline of the shape to form an irregular shape that corresponds to the spatial extent of an irregularly shaped region of vision loss. FIGS. 4A and 4B show an illustrative example of an irregular shape 40 defined by an outline 42 which can be manipulated via user input. In a preferred embodiment, the outline 42 of the shape 40 can be manipulated by using a computer mouse. In this embodiment, the system is configured such that the user can click on any point of the outline 42 and drag that point to a new location to manipulate the shape of the outline 42. For instance, the user can click on point 46a shown in FIG. 4A and drag the point to point 46b shown in FIG. 4B, which shows a dashed line representing the previous location of a portion of the outline 42 that has been moved to the new location as indicated by the solid line on which point 46b lies. As shown in FIG. 4B, the system is configured such that when a point 46a on the outline 42 is moved, adjacent portions of the outline 42 on either side of the point 46a move with the point to new location 46b. These adjacent portions of line are curved to form a smoothed shape without sharp points. This allows the user to intuitively manipulate the outline 42 to form an irregular shape 40 that approximates the shape of the region of vision loss perceived by the user. The user preferably forms the irregular shape 40 so that the shape forms an outline 42 around the region of vision loss wherein the outline generally conforms to the boundaries of the region of vision loss but is generally visible to the user.

Once the shape 40 is set, the quantitative spatial metric 24 is calculated based on the dimensions of the shape 40. In one embodiment, the spatial metric 24 is a measurement of the area of the shape 40. In another embodiment, the spatial metric 24 is a measurement of a distance 44 from the target point 14 to the point of the outline 42 of the shape 40 that is farthest from the target point 14, as shown in FIGS. 4A and 4B. In other embodiments, the metric 24 may be a measurement of a mean length of a plurality of radii extending from the target point 14 to the boundary 42 of the shape 40 measured at angular increments such as one-degree increments. Other measurements of the size of the shape 40, such as the perimeter, may also be used.

FIG. 5 shows an illustrative example in which the target point 14 is positioned outside of the irregular shape 40 formed by the user to correspond to the region of vision loss. Regardless of the final shape selected by the user, the system is preferably configured such that the size of the shape 40 can be varied by the user while maintaining the same shape of the outline 42 of the irregular shape 40. A size adjustment bar 70 may be configured, for instance, to increase the size of the shape 40 such that outline 42, which corresponds to the region of vision loss, is expanded to outline 43, which extends outside the region of vision loss perceived by the user. Thus, the area 58 located between outline 42 and outline 43 is visible to the user. The color adjustment bar 26 may then be used to change the color of the shape until the color of area 58 approximates the color of the region of vision loss.

Another aspect of the disclosure includes a system configured for assessing central or paracentral vision loss. In one embodiment, the system comprises a display screen 12 and a computer 10 operably connected to the display screen 12. The computer 10 is configured to present a shape to a user on the display screen 12 and to allow the user to manipulate the dimensions of the shape via user input such that the shape corresponds to the spatial extent of a region of vision loss perceived by the user. The computer 10 is further configured to calculate a quantitative spatial metric 24 indicating the spatial extent of the region of vision loss based on a measurement of the size of the shape. The computer 10 may also be configured to allow the user to vary the value of the color of the shape via user input and to calculate a quantitative color metric 24 indicating the degree of visual impairment within the region of vision loss based on the color value. The computer 10 may have an executable program installed thereon for carrying out the functions of the methods disclosed herein, or the program may be accessed remotely via the internet by accessing a website configured for administering a vision assessment test in accordance with the present methodology. The system may also comprise a database server operably connected directly to the computer 10 or accessed remotely via the internet. The database server is used for storing and retrieving quantitative results and other data for vision assessment tests.

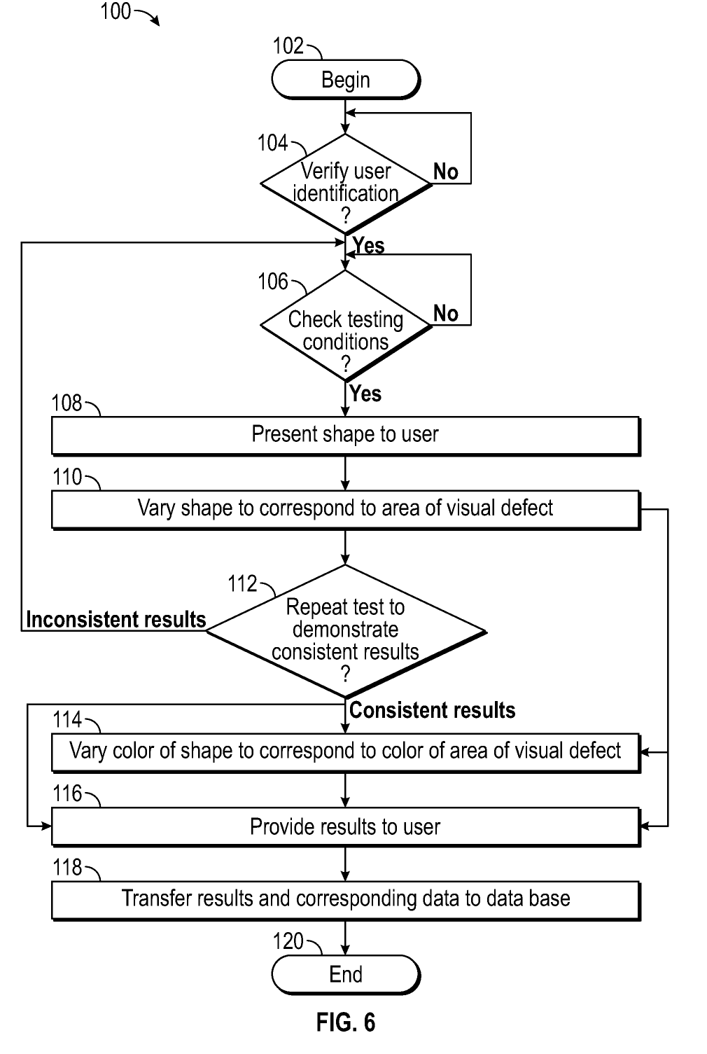

FIG. 6 illustrates a flow chart 100 showing some possible steps of a vision assessment test that may be carried out by a computer executable program in accordance with the present methodology. Step 102 indicates the beginning of the vision assessment method. Before beginning a vision assessment for the first time, a user may input user identification information and set up a user account, which may include a username and password or other similar types of identity verification information. In control step 104, the user inputs the identity verification information, which may include a password, passcode, biometric information such as fingerprints, or similar types of identifying information. After the user's identity has been verified, testing conditions may be evaluated in step 106. Step 106 may involve checking that the user is positioned at an appropriate viewing distance 15 from the display screen 12 in order to maintain consistent results in the assessment. The camera 13 may be utilized in conjunction with the executable program for this purpose. The program may also be configured to calibrate the system to set an appropriate distance 15 before beginning an assessment for the first time and to verify the correct distance on subsequent assessments. The camera 13 and program may also be configured to verify that the lighting is sufficient for administering the test.

Next, in step 108 the system presents a shape to the user on the display screen 12. Step 108 may be initiated automatically by the system or may be initiated by the user. In step 110, the user then manipulates the dimensions of the shape via user input to correspond to the spatial extent of a region of vision loss perceived by the user, and the program changes the dimensions of the shape according to the user input. When viewing and manipulating the shape, the user should close or cover one eye so that the shape is visible by only one eye. The test may then be repeated with the other eye closed or covered. Resulting data may be obtained separately for independently assessing the vision of both the left and right eyes. User input controls are provided for manipulating the dimensions of the shape and may be in the form of software, hardware, or firmware components. Once a shape is selected that corresponds to a region of the user's vision loss, the program is configured to allow the user to confirm the finalized shape to be used for determining the test results. A quantitative spatial metric is then calculated and displayed to the user. In optional step 112, the assessment test may be repeated to demonstrate that consistent results are obtained.

In accordance with one embodiment, the flow chart 100 may then proceed to step 116 in which final results are provided to the user. In a preferred embodiment, step 114 is performed before step 116. In step 114, the user varies the value of the color of the shape to correspond to the color of the region of vision loss as perceived by the user. When the selected color is finalized and confirmed by the user, a quantitative color metric is then calculated and displayed to the user. In this embodiment, the final results provided to the user in step 116 include both the spatial metric and the color metric. Step 116 may also involve comparing the results of the assessment with results obtained in any previous tests, display results of previous tests for comparison, and notifying the user if a significant change, which may be defined as a certain percentage change in the numeric value of a metric, has occurred in either metric. In step 118, the final results are transferred to a database for storage and future retrieval. The database may be associated with a user account, in which case the results from an assessment will be compiled with the results of any previous assessments to create a quantitative record of changes to the user's vision. Any corresponding data obtained from the assessment, which may include data such as date stamps and time stamps for the results, are transferred to the database with the final results. Thus, the quantitative record comprises a set of quantitative spatial metrics and corresponding quantitative color metrics, including additional optional data. All of the data may be uploaded to a database server via the internet. Preferably, the user's eye doctor may also access this information, and the program may be configured to send the results to the user's doctor and notify the doctor if any significant changes have occurred compared to previous assessments.

Once all of the steps of the vision assessment have been completed, operation ends at step 120, which may include the user logging out of a user account.

It will be readily understood to those skilled in the art that various other changes in the details, material, and arrangements of the parts and method stages which have been described and illustrated in order to explain the nature of this inventive subject matter can be made without departing from the principles and scope of the inventive subject matter.

1. A method of assessing central or paracentral vision loss, said method comprising the steps of:
presenting a shape to a user on a display screen, wherein the display screen is operably connected to a computer,
allowing the user to manipulate the dimensions of the shape via user input, wherein the dimensions of the shape are manipulated by the user such that the shape corresponds to the spatial extent of a region of vision loss of the user,
changing the dimensions of the shape according to the user input, and
calculating a quantitative spatial metric indicating the spatial extent of the region of vision loss based on a measurement of the size of the shape.

2. The method of claim 1, wherein the dimensions of the shape are manipulated by varying the size of the shape.

3. The method of claim 2, wherein the shape is a circle.

4. The method of claim 1, wherein the dimensions of the shape are manipulated by varying the dimensions along two axes.

5. The method of claim 4, wherein the shape is an ellipse.

6. The method of claim 1, wherein the dimensions of the shape are manipulated by manipulating the outline of the shape to form an irregular shape.

7. The method of claim 6, further comprising the step of allowing the user to vary the size of the newly formed irregular shape.

8. The method of claim 1, further comprising the step of storing the quantitative spatial metric in a database.

9. The method of claim 8, further comprising the step of repeating the steps of said method at spaced time intervals such that the quantitative spatial metric for each iteration is stored in the database to create a quantitative record of changes to the user's vision over the duration of the spaced time intervals.

10. The method of claim 1, wherein the shape has a color, said method further comprising the steps of:
allowing the user to vary the value of the color of the shape via user input, wherein the color value of the shape is varied by the user such that the color of the shape corresponds to the color of the region of vision loss as perceived by the user,
varying the value of the color of the shape according to the user input, and
calculating a quantitative color metric indicating the degree of visual impairment in the region of vision loss based on the color value of the shape.

11. The method of claim 10, further comprising the step of storing the quantitative spatial metric and the quantitative color metric in a database.

12. The method of claim 11, further comprising the step of repeating the steps of said method at spaced time intervals such that the quantitative spatial metric and the quantitative color metric for each iteration is stored in the database to create a quantitative record of changes to the user's vision over the duration of the spaced time intervals.

13. The method of claim 10, wherein the color of the shape is gray.

14. A system for assessing central or paracentral vision loss, said system comprising a computer and a display screen operably connected to the computer, wherein the computer is configured to present a shape to a user on the display screen and to allow the user to manipulate the dimensions of the shape via user input such that the shape corresponds to the spatial extent of a region of vision loss of the user, wherein the computer is further configured to calculate a quantitative spatial metric indicating the spatial extent of the region of vision loss based on a measurement of the size of the shape.

15. The system of claim 14, wherein the computer is configured to allow the user to manipulate the dimensions of the shape by varying the size of the shape.

16. The system of claim 14, wherein the computer is configured to allow the user to manipulate the dimensions of the shape by varying the dimensions of the shape along two axes.

17. The system of claim 14, wherein the computer is configured to allow the user to manipulate the dimensions of the shape by manipulating the outline of the shape to form an irregular shape.

18. The system of claim 17, wherein the computer is further configured to allow the user to vary the size of the newly formed irregular shape.

19. The system of claim 14, wherein the computer is further configured to store the quantitative spatial metric in a database.

20. The system of claim 19, wherein the computer is further configured to create a quantitative record of changes to the user's vision, wherein the quantitative record comprises a set of quantitative spatial metrics recorded at spaced time intervals.

21. The system of claim 14, wherein the shape has a color, wherein the computer is further configured to: allow the user to vary the value of the color of the shape via user input such that the color of the shape corresponds to the color of the region of vision loss as perceived by the user, and to calculate a quantitative color metric indicating the degree of visual impairment in the region of vision loss based on the color value of the shape.

22. The system of claim 21, wherein the computer is further configured to store the quantitative spatial metric and the quantitative color metric in a database.

23. The system of claim 22, wherein the computer is further configured to create a quantitative record of changes to the user's vision, wherein the quantitative record comprises a set of quantitative spatial metrics and corresponding quantitative color metrics recorded at spaced time intervals.
